(19) United States
(12) Patent Application Publication
(10) Pub. No.: US 2024/0125757 A1
(43) Pub. Date: Apr. 18, 2024

(54) DEVICE AND METHOD FOR DETECTING SOIL NUTRIENTS ON SITE AND MICROFLUIDIC CHIP

(71) Applicant: ZHONGKE HEFEI INSTITUTES OF INTELLIGENT AGRICULTURE, Hefei (CN)

(72) Inventors: Rujing WANG, Hefei (CN); Yongjia CHANG, Hefei (CN); Xiangyu CHEN, Hefei (CN); Qinwen LU, Hefei (CN); Jiangning CHEN, Hefei (CN); Qiao CAO, Hefei (CN); Yang LIU, Hefei (CN); Xiaoyu ZHANG, Hefei (CN); Jiahao XIAO, Hefei (CN); Hongyan GUO, Hefei (CN); Dapeng WANG, Hefei (CN)

(21) Appl. No.: 18/396,333

(22) Filed: Dec. 26, 2023

Related U.S. Application Data
(63) Continuation of application No. PCT/CN2022/133847, filed on Nov. 23, 2022.

(30) Foreign Application Priority Data
Jun. 13, 2022 (CN) 202210659264.2
Jun. 13, 2022 (CN) 202210659309.6

(51) Int. Cl. G01N 33/24; G01N 1/40; G01N 21/64; G01N 27/04

(52) U.S. Cl. CPC G01N 33/24; G01N 1/4077; G01N 21/643; G01N 27/043; G01N 27/048; G01N 2033/245

(57) ABSTRACT

A device for detecting soil nutrients on site, including an extracting grid, an on-site real-time detection assembly and a transfer assembly for transferring a soil extract from the extracting grid to the on-site real-time detection assembly. A soil nutrient detection method using the device and a microfluidic chip are also provided. The microfluidic chip includes a cover plate and a base plate. The base plate includes a soil extract feeding groove, a quantitative feeding groove, a reagent storage groove, and a serpentine groove.

### CROSS-REFERENCE TO RELATED APPLICATIONS

This application is a continuation of International Patent Application No. PCT/CN2022/133847, filed on Nov. 23, 2022, which claims the benefit of priority from Chinese Patent Applications No. 202210659309.6, filed on Jun. 13, 2022, and No. 202210659264.2, filed on Jun. 13, 2022. The content of the aforementioned application, including any intervening amendments thereto, is incorporated herein by reference in its entirety.

### TECHNICAL FIELD

This application relates to soil nutrient detection, in particular to a device and a method for detecting soil nutrients on site and a microfluidic chip.

### BACKGROUND

In order to increase production, agricultural producers often blindly apply excessive fertilizer to the soil, such that the current agricultural production in China is struggling with increasing fertilizer without increasing yield and low nutrient utilization efficiency. Moreover, a large amount of the fertilizer will lead to water pollution and soil pollution, which should not be overlooked. During the agricultural production, accurate management of soil nutrients and rational fertilization for improving use efficiency of nutrients are not only an effective way to improve grain production and reduce environmental pollution, but also urgent requirements to ensure food security and sustainable agricultural development in China. Accurate management of soil nutrients is of great significance for increasing agricultural production while decreasing fertilizer usage and promoting environmental protection in China. Additionally, the determination for available contents of nitrogen (N), phosphorus (P) and potassium (K) in soil is the cornerstone of accurate management of soil nutrients.

In the existing technologies, the traditional method for detecting the available contents of N, P and K in soil mainly include performing field sampling investigation, collecting a soil sample and subjecting the soil sample to air drying and grinding in the laboratory, detecting the contents of nutrient ions in the soil sample in combination with laboratory analysis (methods such as a flame spectrophotometry method, a flow injection analyzer method and a total nitrogen digestion method). Although these methods have high accuracy in detecting contents of soil nutrient ions, the corresponding detection have cumbersome process, and consumes much manpower, materials and financial resources. Moreover, these methods often have detection delay.

At present, regarding the rapid detection of the available contents of N, P and K in the soil, different methods and devices are employed for detecting different elements, which leads to long detection time, complicated operation, and large consumption for various types of chemical reagents. The traditional method for detecting the soil sample is time-consuming, which makes it unsuitable for immediate variable fertilization. Therefore, it is crucial for precision agriculture and agricultural development to determine the available contents of N, P and K in soil rapidly and accurately.

In order to achieve rapid detection for the available contents of N, P and K in soil, the multi-element synchronous and continuous detection technology is studied and developed. Moreover, the microfluidic combined with fluorescence detection technology is also developed to make the rapid detection possible.

In the microfluidic technology, the micron-scale microchannels are constructed to realize the controlled delivery of micro liquid. The microfluidic technology has the advantages of small sample demand, fast mass transfer, easy portability, simultaneous multi-channel detection of multiple samples, and easy integration with optical and other detection methods. Moreover, the microchip manufactured by the microfluidic technology has extremely low material cost, and can encapsulate trace of reaction reagents in advance. In addition, the microchip and the reaction reagents cause no secondary pollution to the environment.

However, less attention has been paid to how to realize the rapid and accurate detection of soil nutrients on site by using microfluidic technology combining with fluorescence technology. Therefore, it is urgently required to develop a device and method for rapidly and accurately acquiring information about the available contents of N, P and K in soil in the field. The device and method are required to be simpler for agricultural producers to operate without complicated training, and are convenient for being promoted, so as to guide variable fertilization. Meanwhile, a centrifugal microfluidic chip with high automation, high efficiency, simplicity, accuracy and rapidity is also needed to realize the detection of soil ions on site.

### SUMMARY

An objective of this application is to provide a device and method for detecting soil nutrients on site and a microfluidic chip to remedy the insufficiency in the existing rapid and accurate detection of available contents of N, P and K in soil on site.

Technical solutions of this application are described as follows.

In a first aspect, this application provides a device for detecting soil nutrients on site, comprising:
an extracting grid;
an on-site real-time detection assembly; and
a transfer assembly;
wherein the transfer assembly is configured to transfer a fresh soil extract sample from the extracting grid to the on-site real-time detection assembly;
the on-site real-time detection assembly comprises a driving motor assembly and a detection-analysis assembly, wherein an output shaft of the driving motor assembly is provided with a microfluidic soil chip;
the microfluidic soil chip comprises a base plate; a bottom of the base plate is provided with an aligning slot; the microfluidic soil chip is mounted on the output shaft of the driving motor assembly through the aligning slot; an upper surface of the base plate is located in a housing of the microfluidic soil chip; a center of the upper surface of the base plate is etched with a soil extract feeding groove; a first flow channel region, a second flow channel region, a third flow channel region and a fourth flow channel region are sequentially provided on the base plate from the soil extract feeding groove to outside; and the first flow channel region, the second flow channel region, the third flow channel region and the fourth flow channel region are the same in structure.

In an embodiment, the first flow channel region comprises a quantitative feeding groove for quantitative feeding of a soil extract and a reagent storage groove; the quantitative feeding groove is connected to the soil extract feeding groove through a first microchannel; an outlet of the reagent storage groove is connected to a T-shaped mixing groove through a second microchannel; an outlet of the quantitative feeding groove is connected to the T-shaped mixing groove through a third microchannel; the T-shaped mixing groove is connected to a mixing-zone groove; an outlet of the mixing-zone groove is connected to a detection-zone groove through a serpentine groove; a fluorescence exciter and a receiver are located on a rotation trajectory of the detection-zone groove; and
a width of an end of the second microchannel connected with the T-shaped mixing groove is smaller than a width of an end of the second microchannel connected with the outlet of the reagent storage groove; a width of an end of the third microchannel connected with the T-shaped mixing groove is smaller than a width of an end of the third microchannel connected with the outlet of the quantitative feeding groove;
the serpentine groove is connected to the detection-zone groove through a fourth microchannel, and a width of the fourth microchannel is smaller than the width of the end of the third microchannel connected with the outlet of the quantitative feeding groove.

In an embodiment, the detection-analysis assembly comprises a control processor, an acquisition card, a fluorescence exciter, a receiver and a soil moisture-temperature-electric conductivity sensor; the fluorescence exciter is connected to a first control signal output end of the control processor; the receiver is connected to a first data input end of the control processor through the acquisition card; a data output end of the soil moisture-temperature-electric conductivity sensor is connected to a second data input end of the control processor; and the driving motor assembly is connected to a second control signal output end of the control processor.

In an embodiment, a sealing film is attached to an upper surface of the extracting grid; an extracting reagent is contained in the extracting grid; the number of the extracting grid is n; and any two adjacent extracting grids are connected to each other through a mortise-tenon structure.

In an embodiment, the transfer assembly comprises a negative-pressure suction bag; a rear end of the negative-pressure suction bag is provided with a quick-detachable connector; a front end of the negative-pressure suction bag is provided with a quantitation loop; a front end of the quantitation loop is provided with a suction head; and a filter block is provided in the suction head.

In an embodiment, a reagent storage groove of the first flow channel region is configured to store a specific potassium detection reagent; a reagent storage groove of the second flow channel region is configured to store a specific ammonia-nitrogen detection reagent; a reagent storage groove of the third flow channel region is configured to store a specific nitrate (NO₃⁻) detection reagent; and a reagent storage groove of the fourth flow channel region is configured to store a specific phosphorus detection reagent.

In an embodiment, the base plate is circular; the soil extract feeding groove, the quantitative feeding groove, the reagent storage groove and the mixing-zone groove are circular; and the first flow channel region and the third flow channel region are located on a horizontal axis of the base plate, and the second flow channel region and the fourth flow channel region are located on a vertical axis of the base plate.

In an embodiment, the negative-pressure suction bag and the quick-detachable connector are made of a flexible plastic material; the quantitation loop and the suction head are made of a rigid plastic material; and the filter block is made of filter cotton or quartz sand.

In an embodiment, the microfluidic soil chip further comprises a cover plate located on the base plate.

In an embodiment, the cover plate comprises a main body and a quantitative feeding hole of the fresh soil extract sample; the quantitative feeding hole is provided on a middle of the main body; and the quantitative feeding hole is communicated with the soil extract feeding groove;
the main body is provided with a guiding groove of the fresh soil extract sample, a detection reagent feeding hole and a window; the number of the guiding groove, the number of the detection reagent feeding hole, the number of the window and the number of the first flow channel region, the second flow channel region, the third flow channel region and the fourth flow channel region are the same the detection reagent feeding hole is communicated with mixing-zone grooves of the first flow channel region, the second flow channel region, the third flow channel region and the fourth flow channel region in one-to-one correspondence; the window is arranged above detection-zone grooves of the first flow channel region, the second flow channel region, the third flow channel region and the fourth flow channel region in one-to-one correspondence; one end of the guiding groove is communicated with the quantitative feeding hole, and the other end of the guiding groove is provided with a storage zone; and a side of the storage zone is provided with an air hole communicated with the storage zone.

In a second aspect, this application provides a method for detecting soil nutrients on site by using the device mentioned above, comprising:
(S91) obtaining a linear relationship curve between soil concentration and fluorescence intensity;
(S92) inserting a soil moisture-temperature-electric conductivity sensor into a field to be analyzed, and acquiring, by a control processor, moisture-temperature-electric conductivity information; wherein the moisture-temperature-electric conductivity information includes a moisture content t with a unit of %, temperature T with a unit of ° C. and electric conductivity with a unit of mS/cm;
(S93) collecting a fresh soil sample; transferring the fresh soil sample to the extracting grid followed by extraction with an extracting reagent under shaking for 3-5 min to obtain a soil extract sample; wherein a weight ratio of the fresh soil sample to the extracting reagent is 1:5;
(S94) transferring the soil extract sample from the extracting grid to the soil extract feeding groove of the microfluidic soil chip by using the transfer assembly;
(S95) controlling, by the control processor, the driving motor assembly to work to drive the microfluidic soil chip to perform rotating centrifugal motion, so as to allow centrifugal decomposition of the soil extract sample in the microfluidic soil chip;
(S96) controlling, by the control processor, a fluorescence exciter and a receiver to work to acquire fluorescence data of the soil extract sample in a detection-zone groove on the microfluidic soil chip; finding a soil concentration c corresponding to the fluorescence data according to the linear relationship curve; and
(S97) according to the moisture-temperature-electric conductivity information and the soil concentration c, calculating, by the control processor, soil nutrient content with a unit of mg/kg based on the following equation:

Xi=5*c/(1−t);
wherein Xi indicates a nitrogen level, phosphorus level or potassium level; 5 is a coefficient, indicating that a weight ratio of the fresh soil sample to water in the extracting grid is 1:5; and c is the soil concentration; and t represents moisture content.

In an embodiment, in step (S95), the centrifugal decomposition is performed through steps of:
(S101) performing a centrifugation at a first rotating speed to allow the soil extract sample to uniformly flow into the first flow channel region, the second flow channel region, the third flow channel region and the fourth flow channel region from the soil extract feeding groove;
(S102) performing a centrifugation at a second rotating speed to allow the soil extract sample and a reagent to pass through a T-shaped mixing groove to enter a mixing-zone groove for uniform mixing and reaction to obtain a reaction mixture; wherein the second rotating speed is higher than the first rotating speed;
(S103) subjecting the reaction mixture to standing to allow complete reaction, and allowing the reaction mixture to enter a serpentine groove for further mixing and reaction; and
(S104) performing a centrifugation at a third rotating speed to allow the reaction mixture to pass through the serpentine groove and enter the detection-zone groove; wherein the third rotating speed is higher than the second rotating speed.

In a third aspect, this application provides a microfluidic chip, comprising:
a cover plate and a base plate arranged in sequence;
wherein the base plate comprises a main body, a soil extract feeding groove and a plurality of channel branches; the soil extract feeding groove is provided on a middle portion of a top of the main body of the base plate; the plurality of channel branches are provided on the top of the main body of the base plate, and are uniformly distributed along a periphery of the soil extract feeding groove; the plurality of channel branches comprise a quantitative feeding groove, a reagent storage groove, a mixing-zone groove, a serpentine groove and a detection-zone groove; the quantitative feeding groove is communicated with the soil extract feeding groove; a T-shaped mixing groove is arranged between the quantitative feeding groove and the reagent storage groove; the quantitative feeding groove and the reagent storage groove are connected through the T-shaped mixing groove, and then connected to one end of the mixing-zone groove; the other end of the mixing-zone groove is communicated with one end of the serpentine groove, and the other end of the serpentine mixing-zone groove is connected to the detection-zone groove through a capillary micro-valve.

In an embodiment, the cover plate comprises a main body and a quantitative feeding hole of the fresh soil extract sample; wherein the quantitative sample-feeding hole is arranged on a middle portion of the main body of the cover plate, and is communicated with the soil extract feeding groove; and

the main body of the cover plate is also provided with a guiding groove of the fresh soil extract sample, a detection reagent feeding hole and a window, wherein the number of the guiding groove of the fresh soil extract sample, the detection reagent feeding hole, the window and the plurality of channel branches are the same in quantity; the detection reagent feeding hole is arranged in one-to-one correspondence with a mixing-zone groove; the detection reagent feeding hole is communicated with a corresponding mixing-zone groove; the window is arranged in one-to-one correspondence with a detection-zone groove; the window is located above a corresponding detection-zone groove; one end of the guiding groove of the fresh soil extract sample is communicated with the quantitative sample-feeding hole for the fresh soil extract sample, and the other end of the guiding groove of the fresh soil extract sample is provided with a storage zone; and a side of the storage zone is provided with a first air hole; and the first air hole is communicated with the storage zone.

In an embodiment, the serpentine groove has a spiral shape, or is formed by a plurality of continuous zigzags

In an embodiment, a middle of a back of the main body of the base plate is provided with a chip fixing hole.

In an embodiment, a side of the detection-zone groove is provided with a second air hole communicated with the detection-zone groove; and the second air hole is communicated with the first air hole in one-to-one correspondence.

In an embodiment, the window comprises a through hole and an optically-transparent film; the through hole is provided on the main body of the cover plate; and the optically-transparent film is provided in the through hole.

In a fourth aspect, this application provides a method for detecting soil nutrients on site by using the microfluidic chip mentioned above, comprising:
(S1) installing the microfluidic chip on a centrifugal detector;
(S2) injecting the fresh soil extract sample into the quantitative feeding hole; starting the centrifugal detector to drive the microfluidic chip to rotate at a first rotation speed for T1 seconds; wherein during rotation of the microfluidic chip, the fresh soil extract sample flows from the quantitative feeding hole sequentially to the soil extract feeding groove and the quantitative feeding groove; and excess fresh soil extract sample in the quantitative feeding hole flows along the guiding groove to the storage zone;
(S3) driving, by the centrifugal detector, the microfluidic chip to rotate at a second rotating speed for T2 seconds, wherein the second rotating speed is larger than the first rotating speed; during rotation of the microfluidic chip, the fresh soil extract sample flows from the quantitative feeding groove to the mixing-zone groove through the T-shaped mixing groove, and a detection reagent flows from the reagent storage groove to the mixing-zone groove through the T-shaped mixing groove; and the fresh soil extract sample is mixed and reacted with the detection reagent in the mixing-zone groove to obtain a reaction mixture;
(S4) stopping the centrifugal detector to allow the reaction mixture to flow from the mixing-zone groove to the serpentine groove to allow further mixing and reaction of the fresh soil extract sample and the detection reagent in the reaction mixture in the serpentine groove, and flow to the capillary micro-valve; and
(S5) restarting the centrifugal detector to drive the microfluidic chip to rotate at a third rotation speed for T3 seconds to allow the reaction mixture to pass through the capillary micro-valve to flow into the detection-zone groove.

In an embodiment, the number of the reagent storage groove is two or more; and detection reagents for detecting different ions are respectively pre-stored in two or more reagent storage grooves.

Compared with the prior art, this application has the following beneficial effects.

The device for detecting soil nutrients on site provided herein employs synchronous and continuous detection of multiple elements to realize the effective and rapid detection of available contents of N, P and K in soil, and then uses the microfluidic technology combining with fluorescence detection analysis technology on the basis of analysis of the microfluidics and specific fluorescent quantum dots to achieve the direct detection for fresh soil contents on site.

With respect to the microfluidic chip provided herein, the reaction is driven and controlled under the centrifugal force, which realizes the synchronous detection of various soil ions, thereby reducing the manual participation, simplifying the detection and improving the efficiency and accuracy of the detection.

This application realizes the rapid and detection for soil contents on site with high accuracy and high integration level. In this application, multiple indexes of nitrogen, phosphorus and potassium can be detected synchronously, which reduces the manual participation, improving the automation degree, simplifying the operation of the detection and making the detection accurate and fast.

### BRIEF DESCRIPTION OF THE DRAWINGS

FIG. 1 is a schematic diagram of a structure of a device for detecting soil nutrients on site and a microfluidic chip according to an embodiment of this application;

FIG. 2 is a schematic diagram of a structure of an extracting grid in FIG. 1 according to an embodiment of this application;

FIG. 3 is a schematic diagram of a structure of a transfer assembly in FIG. 1 according to an embodiment of this application;

FIG. 4 is a schematic diagram of a structure of an on-site real-time detection assembly in FIG. 1 according to an embodiment of this application;

FIG. 5a is a perspective view of a base plate 101 of a microfluidic chip in FIG. 1 according to an embodiment of this application;

FIG. 5b is a structural diagram of a cover plate 102 matched with the base plate in FIG. 5 (4 flow channel regions);

FIG. 6 is a flow chart of a method for detecting soil nutrients on site and a microfluidic chip according to an embodiment of this application

FIG. 7 is a stereo diagram of a microfluidic chip in Example 2 of this application (5 channel branches);

FIG. 8 is a structural diagram of the cover plate 102 of the microfluidic chip in FIG. 7 (5 channel branches);

FIG. 9 is a structural diagram of the base plate 101 of the microfluidic chip ub FIG. 7 (five channel branches).

In the drawings, 1, on-site real-time detection assembly; 2, transfer assembly; 3, extracting grid; 4, soil moisture-temperature-electric conductivity sensor; 5, acquisition card; 6, receiver; 7, fluorescence exciter; 8, control processor; and 9, driving motor assembly;

10, microfluidic chip; 101, base plate; 102, cover plate; 1021, quantitative feeding hole; 1022, guiding groove; 1023, detection reagent feeding hole; 1024, first air hole; 1025, window; and 1026, storage zone;

11, detection-zone groove; 12, feeding groove; 13, aligning slot; 14, serpentine groove; 15, mixing-zone groove; 16, T-shaped mixing groove; 17, second air hole; 18, quantitative feeding groove; 19, microchannel; and 20, reagent storage groove;

21, quick-detachable connector; 22, negative-pressure suction bag, 23, quantitation loop; and 24, filter block; and

29, capillary micro-valve II.

### DETAILED DESCRIPTION OF EMBODIMENTS

This application will be described in detail below with reference to the accompanying drawings and the following embodiments.

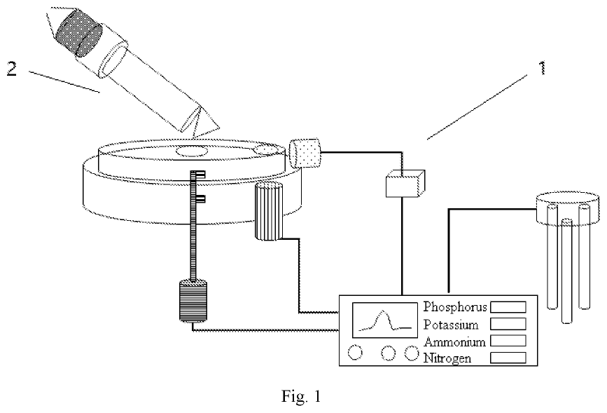

Referring to an embodiment shown in FIG. 1, this application provides a device for detecting soil nutrients on site, which includes an extracting grid 3, an on-site real-time detection assembly 1 and a transfer assembly 2. The transfer assembly 2 is configured to transfer a fresh soil extract sample from the extracting grid 3 to the on-site real-time detection assembly 1.

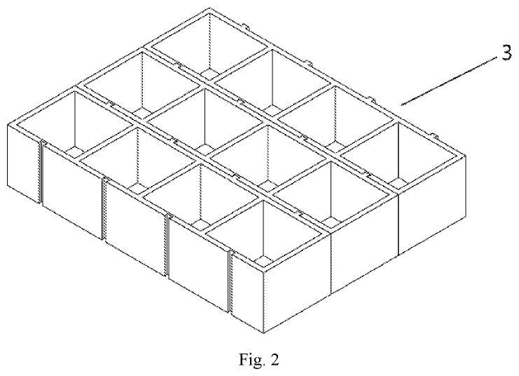

As shown in FIG. 2, the extracting grid 3 is designed to realize the collection and treatment of the soil in the agricultural field. A sealing film is attached to an upper surface of the extracting grid. The extracting reagent is combined in the extracting grid. In practical use, the fresh soil at a surface of the field is collected, and the gravel in the collected soil are removed. Then, the soil can be directly poured into the extracting grid 3. The number of the extracting grid 3 is multiple. There are a plurality of extracting grids 3. The adjacent extracting grids 3 are installed and connected to each other through a mortise-tenon structure, facilitating the use and realizing the portability. That is to say, four extracting grids are arranged in a horizontal row. The horizontal rows are easily expanded through a mortise-tenon structure. Each of the plurality of extracting grids is added with soil extracting agent in advance, and covered and sealed by polyvinyl chloride (PVC) film. During on-site use, agricultural producers select and form the number of the extracting grids according to the number of soil samples to be detected on the same day, uncover the PVC film (sealing film), weigh the soil samples and put the soil samples into the extracting grids 3 in sequence, and then cover the rigid plastic material (such as polypropylene (PP)) box lid to shake the soil samples uniformly, so as to increase the extracting efficiency. In this way, the field extracting treatment of the soil is realized without needing to bring the soil samples back to the laboratory for extraction treatment. Moreover, the soil samples are fresh soil, and thus the obtained data of soil nutrients is more accurate.

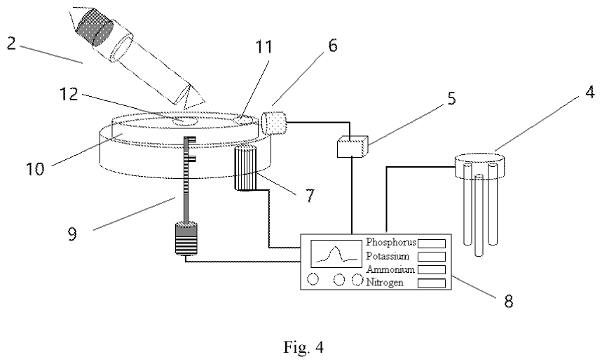

As shown in FIG. 4, the on-site real-time detection assembly 1 includes a driving motor assembly 9 and a detection-analysis assembly. An output shaft of the driving motor assembly 9 is provided with a microfluidic chip 10. That is, the microfluidic chip 10 is installed on the output shaft of the driving motor assembly 9 in a traditional way. The driving motor assembly 9 drives the microfluidic chip 10 to perform centrifugal rotation.

The detection-analysis assembly includes a control processor 8, an acquisition card 5, a fluorescence exciter 7, a receiver 6 and a soil moisture-temperature-electric conductivity sensor 4. The fluorescence exciter 7 is connected to a first control signal output end of the control processor 8. The control processor 8 is configured to control the fluorescence exciter 7 to emit fluorescence signal. The receiver 6 is connected to a first data input end of the control processor 8 through the acquisition card 5. The receiver 6 is configured to acquire fluorescence data for penetrating through the detection-zone groove 11 on the microfluidic chip 10 by the fluorescence exciter 7. A data output end of the soil moisture-temperature-electric conductivity sensor 4 is connected to a second data input end of the control processor 8. The driving motor assembly 9 is connected to a second control signal output end of the control processor 8. In this case, through the design of the soil moisture-temperature-electric conductivity sensor 4, the soil moisture-temperature-electric conductivity sensor 4 is configured to be inserted into the field to be analyzed, so as to measure temperature, moisture content, conductivity and other data of the soil. That is, the soil moisture data is obtained. The samples being detected herein is fresh soil containing water without being traditionally evaporated in the lab. Consequently, in order to realize the detection of the fresh soil containing water, it is necessary to measure the soil moisture to remove the moisture factor during analyzation.

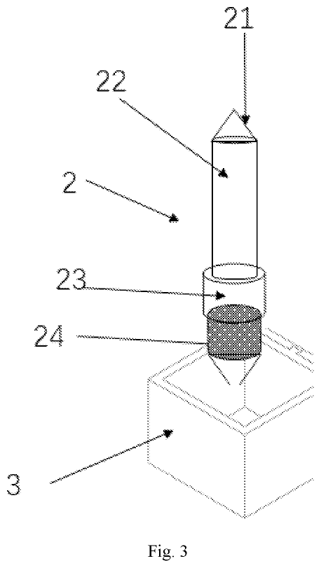

As shown in FIG. 3, the transfer assembly 2 is configured to transfer the fresh soil extract sample. The negative-pressure suction bag 22 is configured to suck the fresh soil extract sample. A rear end of the negative-pressure suction bag 22 is provided with a quick-detachable connector 21, so that the fresh soil extract sample can be conveniently poured out through the quick-detachable connector 21. A front end of the negative-pressure suction bag 22 is provided with a quantitation loop 23. A front end of the quantitation loop 23 is provided with a suction head. A filter block 24 is provided in the suction head. The fresh soil extract sample sucked through the suction head is stored in the quantitation loop 23 after being filtered. The capacity of the quantitation loop 23 is designed according to actual requirements. The negative-pressure suction bag 22 and the quick-detachable connector 21 can be made of flexible plastic material. The quantitation loop 23 and the suction head can be made of rigid plastic material. The filter block 24 can be filter cotton or quartz sand.

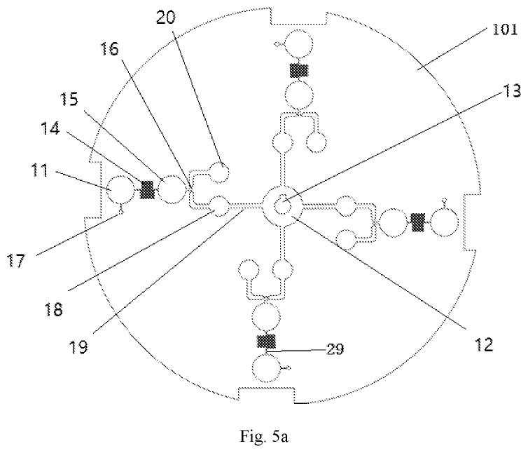

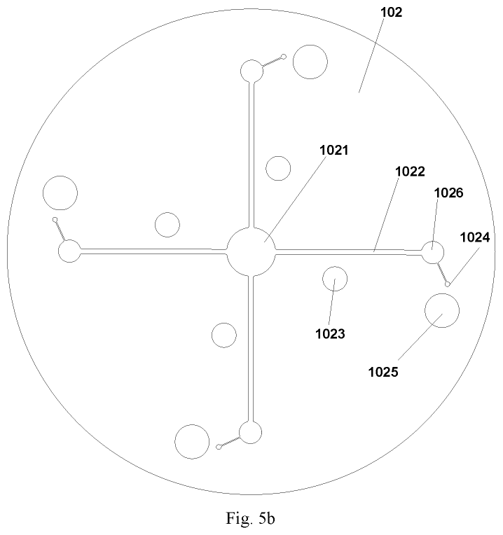

As shown in FIG. 5, the microfluidic chip 10 includes a base plate 101. A bottom of the base plate 101 is provided with an aligning slot 13. The aligning slot 13 is configured for installation of the driving motor assembly 9. The microfluidic chip 10 is installed on an output shaft of the driving motor assembly 9 through the aligning slot 13.

An upper surface of the base plate 101 is located in a housing of the microfluidic chip. A cover plate 102 is provided on the base plate 101. A center of the upper surface of the base plate 101 is etched with a soil extract feeding groove 12. According to the conventional design, the soil extract feeding groove 12 on the housing is designed to be a thin plastic or a structure that is easy to be opened and closed, which helps the quick-detachable connector 21 to pour the fresh soil extract sample into the soil extract feeding groove 12. A first flow channel region, a second flow channel region, a third flow channel region and a fourth flow channel region are sequentially provided on the base plate 101 from the soil extract feeding groove 12 to outside. The first flow channel region, the second flow channel region, the third flow channel region and the fourth flow channel region are the same in structure.

The microfluidic chip 10 further comprises a cover plate 102 located on the base plate 101. The cover plate 102 is in bonding connection to the base plate 101. The cover plate is not shown in FIG. 4. FIG. 5b shows the cover plate 102 corresponding to the base plate 101 in FIG. 5a, which includes four flow channel regions.

The cover plate 102 includes a main body and a quantitative feeding hole 1021 of the fresh soil extract sample provided on a middle of the main body. The quantitative feeding hole 1021 is communicated with the soil extract feeding groove 12. The main body is further provided with a guiding groove 1022 of the fresh soil extract sample, a detection reagent feeding hole 1023 and a window 1025. The number of the guiding groove 1022, the number of the detection reagent feeding hole 1023, the number of the window 1025 and the number of the first flow channel region, the second flow channel region, the third flow channel region and the fourth flow channel region are the same.

The detection reagent feeding hole 1023 is communicated with mixing-zone grooves 15 of the first flow channel region, the second flow channel region, the third flow channel region and the fourth flow channel region in one-to-one correspondence The window 1025 is arranged above a detection-zone grooves 11 of the first flow channel region, the second flow channel region, the third flow channel region and the fourth flow channel region in one-to-one correspondence. One end of the guiding groove 1022 is communicated with the quantitative feeding hole 1021, and the other end of the guiding groove 1022 is provided with a storage zone 1026. A side of the storage zone 1026 is provided with a first air hole 1024. The first air hole 1024 is communicated with the storage zone. The window 1025 includes a through hole and an optically-transparent film installed in the through hole.

The first flow channel region, the second flow channel region, the third flow channel region and fourth flow channel region on the microfluidic chip 10 are four channels respectively configured to detect four indexes including potassium, ammonia, nitrogen, nitrate and phosphorus. A specific potassium detection reagent is stored in the reagent storage groove 20 of the first flow channel region. A specific ammonia, nitrogen detection reagent is stored in the reagent storage groove 20 of the second flow channel region. A specific nitrate (NO₃⁻) detection reagent is stored in the reagent storage groove 20 of the third flow channel region. A specific phosphorus detection reagent is stored in the reagent storage groove 20 of the fourth flow channel region. In this case, the four indexes to be detected are determined by the reagents stored in the four reagent storage grooves. During the processing of the microfluidic chip, the four reagents are respectively added to the four reagent storage grooves in advance. The four reagents are sealed in the microfluidic chip. The microfluidic chip is transported to the field by vacuum packaging. The structure and functional regions of each channel are the same, and are connected by microchannels. The microchannels generally have a height of 100 um.

In order to improve the centrifugal effect, the first flow channel region and the third flow channel region are located on a horizontal axis of the base plate 101, and the second flow channel region and fourth flow channel region are located on a vertical axis of the base plate 101. Taking the first flow channel region as an example, the first flow channel region includes a quantitative feeding groove 18. The quantitative feeding groove 18 is connected to the soil extract feeding groove 12 through a microchannel 19. An outlet of the quantitative feeding groove 18 is connected to the T-shaped mixing groove 16 through a microchannel 19, and an outlet of the reagent storage groove 20 is connected to the T-shaped mixing groove 16 through a microchannels 19. The T-shaped mixing groove 16 is connected a mixing-zone groove 15 through a microchannel 19. An outlet of the mixing-zone groove 15 is connected to the detection-zone groove 11 through the serpentine groove 14. The fluorescence exciter 7 and the receiver 6 are located on a rotation trajectory of the detection-zone groove 11. That is, the fresh soil extract sample flows from the soil extract feeding groove 12 to the quantitative feeding groove 18, and is mixed with reagent in the reagent storage groove 20, and then flows to the detection-zone groove 11 through the T-shaped mixing groove 16 and the serpentine groove 14. The fluorescence exciter 7 and the receiver 6 are located on the rotation trajectories of the four detection-zone grooves 11, and are positioned by conventional sensing techniques. Meanwhile, for better centrifugal effect, the base plate 101 is circular. The soil extract feeding groove 12, the quantitative feeding groove 18, the reagent storage groove 20 and the mixing-zone groove 15 are all circular. For better liquid conductivity, a second air hole 17 is also designed in the detection-zone groove 11. The second air hole 17 is an open through hole.

In order to achieve the centrifugal mixing, a three-stage mixing technology is employed in the microfluidic chip 10. The T-shaped mixing zone is configured for the primary mixing, and the two channels are gradually narrowed at the intersection, which act as a valve to prevent the fresh soil extract sample from flowing to the detection zone when the fresh soil extract sample is added. After passing through the T-shaped mixing-zone under the centrifugal force, the fresh soil extract sample and the reagent are further mixed in the circular mixing zone. Because the microfluidic control is under a laminar flow state, the fresh soil extract sample, the fresh soil extract sample and the reagent need to be fully mixed and reacted and then flow to the serpentine mixing zone through the repeated winding microchannels for further mixing. A slender microchannel at an end of the serpentine mixing zone is configured as a micro-valve.

In this case, a width of an end of the microchannel 19 (between the T-shaped mixing groove 16 and the reagent storage groove 20) connected with the T-shaped mixing groove 16 is smaller than a width of an end of the microchannel (between the T-shaped mixing groove 16 and the reagent storage groove 20) connected with the outlet of the reagent storage groove 20. A width of an end of the microchannel 19 (between the T-shaped mixing groove 16 and the quantitative feeding groove 18) connected with the T-shaped mixing groove 16 is smaller than a width of an end of the third microchannel connected with the outlet of the quantitative feeding groove 18.

At the same time, in order to facilitate the acquisition and improve accuracy of fluorescence data, the lower cover plate of the detection zone has a thick of only 100 micrometers, which not only reduces the loss of fluorescence when penetrating through the microfluidic wall, but also improves the utilization rate of the light intensity compared with the traditional fluorescence detection. The detector aligning zone is laterally chamfered to facilitate detector alignment while reducing wall thickness.

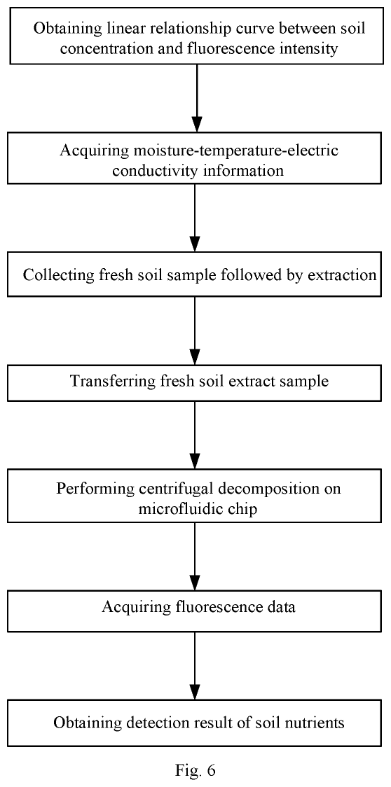

Referring to an embodiment shown in FIG. 6, this application provides a method for detecting soil nutrients on site, which is performed as follows

### (S91) Obtaining of a Linear Relationship Curve Between Soil Concentration and Fluorescence Intensity

The linear relationship curve between soil concentration and fluorescence intensity is obtained. The soil concentration is analyzed by a laboratory. A linear relationship curve between concentrations of four indexes (potassium, ammonia, nitrogen, nitrate nitrogen and phosphorus) of the soil and the fluorescence intensity is the relationship curve determined in the laboratory. That is, soil concentrations are selected, and fluorescence intensity corresponding to the selected soil concentrations are detected, so as to form a linear relationship curve between the soil concentration and the fluorescence intensity. In this case, the approximate soil concentration can be known through the fluorescence intensity. In practical application, when the product is applied in the field, the linear relationship curve is input into the control processor 8.

### (S92) Acquisition of Moisture-Temperature-Electric Conductivity Information

The soil moisture-temperature-electric conductivity sensor 4 is inserted into a field to be analyzed. The control processor 8 acquires the moisture-temperature-electric conductivity information. The moisture-temperature-electric conductivity information includes a moisture content t with a unit of %, temperature with a unit of ° C., and electric conductivity with a unit of mS/cm, that is, the water content information is acquired herein.

### (S93) Collection and Extraction of Fresh Soil Sample

A fresh soil sample is collected. The fresh soil sample is put in the extracting grid 3, and extracted with an extracting reagent under shaking for 3-5 min for extraction to obtain a soil extract sample (fresh soil extract sample). A weight ratio of the fresh soil sample to the extracting reagent in the extracting grid 3 is 1:5.

### (S94) Transferring of the Fresh Soil Extract Sample

The soil extract sample extracted from the extracting grid 3 is transferred to the soil extract feeding groove 12 of the microfluidic chip 10 by using the transfer assembly 2.

### (S95) Centrifugal Decomposition in the Microfluidic Chip 10

The control processor 8 is configured to control the driving motor assembly 9 to drive the microfluidic chip 10 to perform rotating centrifugal rotation, so as to allow centrifugal decomposition of the soil extract sample in the microfluidic chip 10.
(S101) A centrifugation is performed at a first rotating speed to allow the soil extract sample to uniformly flow into the first flow channel region, the second flow channel region, the third flow channel region and the fourth flow channel region from the soil extract feeding groove 12.
(S102) A centrifugation is performed at a second rotating speed to allow the soil extract sample and a reagent to pass through a narrow channel of a T-shaped mixing groove 16 to enter a mixing-zone groove 15. for uniform mixing and reaction to obtain a reaction mixture The second rotating speed is higher than the first rotating speed;
(S103) The reaction mixture is subjected to standing to allow complete reaction. The reaction mixture is allowed to enter a serpentine groove 14 for further mixing and reaction.
(S104) A centrifugation is performed at a third rotating speed to allow the reaction mixture to pass through the serpentine groove 14, and enter a detection-zone groove. The third rotating speed is higher than the second rotating speed.

In the practical application, the above processes are all designed as automatic processes of the driving motor assembly 9, so that the centrifugal rotation is operated automatically without manual operation.

### (S96) Acquisition of Fluorescence Data

The control processor 8 controls the fluorescence exciter 7 and the receiver 6 to work to acquire the fluorescence data of the soil extract sample in the detection-zone groove 11 on the microfluidic chip 10, and finds a soil concentration corresponding to the fluorescence data according to the linear relationship curve.

### (S97) Obtaining of a Detection Result of Soil Nutrients

According to the moisture-temperature-electric conductivity information and the soil concentration c, the control processor 8 calculates soil nutrient content with a unit of mg/kg based on the following equation:

Xi=5*c/(1−t);

where Xi indicates nitrogen level, phosphorus level or potassium level; 5 is a coefficient, indicating that a weight ratio of the fresh soil sample to water in the extracting grid is 1:5; c is the soil concentration; and t represents moisture content.

This application also provides a microfluidic chip 10. The microfluidic chip 10 includes a cover plate 102 and a base plate 101 arranged in sequence.

The base plate 101 includes a main body and a soil extract feeding groove 12 and a plurality of channel branches. The soil extract feeding groove 12 is provided on a middle portion of a top of the main body of the base plate 101. The plurality of channel branches are provided on the top of the main body of the base plate 101, and are uniformly distributed along a periphery of the soil extract feeding groove 12. The plurality of channel branches include a quantitative feeding groove 18, a reagent storage groove 20, a mixing-zone groove 15, a serpentine groove 14 and a detection-zone groove 11. The quantitative feeding groove 18 is communicated with the soil extract feeding groove 12. A T-shaped mixing groove 16 is arranged between the quantitative feeding groove 18 and the reagent storage groove 20. The quantitative feeding groove 18 and the reagent storage groove 20 are connected through the T-shaped mixing groove 16, and then connected to one end of the mixing-zone groove 15. The other end of the mixing-zone groove 15 is communicated with one end of the serpentine groove 14, and the other end of the serpentine groove 14 is connected to the detection-zone groove 11 through a capillary micro-valve II 29.

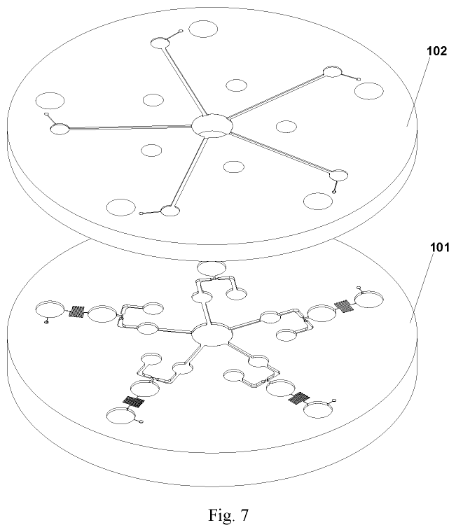

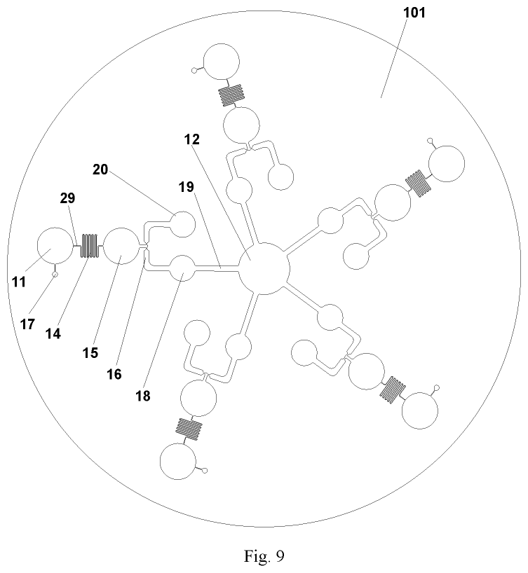

As shown in FIG. 7, the cover plate 102 is in bonding connection to the base plate 101. As shown in FIG. 9, a detection reagent is pre-stored in the reagent storage groove 20. The main body of the base plate 101 includes the soil extract feeding groove 12, a quantitative feeding groove 18, a reagent storage groove 20, a serpentine groove 14, a T-shaped mixing groove 16, a capillary micro-valve II 29, a mixing-zone groove 15, a detection-zone groove 11 and a second air hole 17. The main body of the base plate 101 is uniform in thickness, and is provided on one side of the base plate 101. The chip fixing hole is provided on the other side of the base plate 101 for connecting the microfluidic chip with the centrifugal detector. The chip fixing hole is corresponding to a fixing position structure of the centrifugal detector. The chip fixing hole does not penetrate through the base plate 101 and fails to damage the main body structure on the other side of the base plate 101.

The soil extract feeding groove 12 is configured to allow the base plate 101 to contain the fresh soil extract sample and evenly distribute the fresh soil extract sample to surround channel branches. The quantitative sample feeding groove 18 of the first flow channel region, the quantitative sample feeding groove 18 of the second flow channel region, the quantitative sample feeding groove 18 of the third flow channel region, and the quantitative sample feeding groove 18 of the fourth flow channel region are configured for containing a fixed volume of the fresh soil extract sample and are symmetrically distributed along the soil extract feeding grooves 12 for the fresh soil extract sample, and accurate controlling of the fresh soil extract sample for the reaction under the action of centrifugal driving force. The reagent storage groove 20 is configured to contain a fixed volume of a detection reagent. The detection reagent is a liquid containing a nanoprobe material and is pre-stored in the reagent storage groove 20. A T-shaped mixing groove 16 is provided between the reagent storage groove 20 and the quantitative feeding groove 18 and the mixing-zone groove 15. Two liquids (the fresh soil extract sample and detection reagent) are trapped at a front end of the T-shaped mixing groove 16 before mixing. A middle portion of the T-shaped mixing groove 16 has a capillary structure. The whole of the T-shaped mixing groove 16 forms a capillary micro-valve I. The serpentine groove 14 is configured for improving the mixing and reaction of the fresh soil extract sample and the detection reagent. One end of the serpentine groove 14 is connected with the mixing-zone groove 15, and the reaction mixture of the fresh soil extract sample and the detection reagent which is completely reacted in the mixing-zone groove 15 directly enters the snake-shape mixing-zone groove 14 under the action of capillary force. The other end of the serpentine groove 14 is structurally connected to the detection-zone groove 11 through a capillary micro-valve II 29, and the reaction mixture which is fully mixed and reacted in the serpentine groove 14 is intercepted at the front end of the capillary micro-valve II 29. The T-shaped mixing groove 16 and the capillary micro-valve II 29 are two passive microvalves, and are configured to control the liquid flow in the microfluidic chip. Under the driving of the centrifugal force, the liquid breaks through the capillary microvalve due to internal pressure and enters the next region, so as to realize the working mode of the microfluidic chip driven by the centrifugal force. The T-shaped mixing groove 16 and the capillary micro-valve II 29 are different in structural sizes. The liquid interception of the capillary micro-valve can be broken through by driving under different centrifugal rotating speeds. The fresh soil extract sample and the detection reagent are driven by the centrifugal force to break through the T-shaped mixing groove 16 and enter the mixing-zone groove 15. A flow rate of the liquid is slow under the microfluidic environment. The fresh soil extract sample and the detection reagent are mixed and reacted in the process of filling the mixing-zone groove 15. The reaction mixture of the fresh soil extract sample and the detection reagent after reaction is driven by the centrifugal force to pass through the capillary micro-valve II 29 between the serpentine groove 14 and the detection-zone groove 11, and slowly fill and stay in the detection-zone groove 11 to be detected by fluorescence. The second air hole 17 is communicated with the corresponding first air hole 1024 on the cover plate 102 to maintain the air pressure balance in each of the first, second, third and fourth flow channel region of the microfluidic chip.

The cover plate 102 includes a main body and a quantitative feeding hole 1021. The quantitative feeding hole 1021 is arranged in the middle of the main body, and is communicated with the soil extract feeding groove 12.

The main body of the cover plate 102 is provided with a guiding groove 1022 of the fresh soil extract sample, a detection reagent feeding hole 1023 and a window 1025. The number of the guiding groove 1022, the number of the detection reagent feeding hole 1023, the number of the window 1025 and the number of the plurality of channel branches are the same. The detection reagent feeding hole 1023 is communicated with the mixing-zone groove 15 in one-to-one correspondence. The window is arranged above detection-zone groove 11 in one-to-one correspondence One end of the guiding groove 1022 is communicated with the quantitative feeding hole 1021, and the other end of the guiding groove 1022 is provided with a storage zone 1026. One side of the storage zone 1026 is provided with a first air hole 1024 communicated with the storage zone 1026.

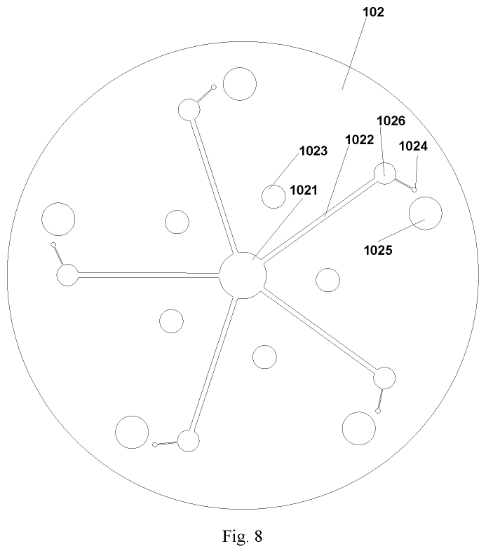

As shown in FIG. 8, the quantitative feeding hole 1021 is the sample injection position of the fresh soil extract sample on the microfluidic chip, and is designed to have but not limited to a cylindrical structure. The quantitative feeding hole 1021 is configured to contain a certain amount of the fresh soil extract sample, and is communicated with the soil extract feeding groove 12 in the base plate 101, so as to allow the liquid to enter the base plate 101. The guiding groove of the first flow channel region, the guiding groove of the second flow channel region, the guiding groove of the third flow channel region and the guiding groove of the fourth flow channel region are uniformly and radially arranged along the quantitative feeding hole 1021. The guiding groove 1022 has a micro-pipeline structure, and is configured to accommodate the excess liquid overflowing from the quantitative feeding hole 1021, and guide the fresh soil extract sample to the storage zone 1026 at a tail end of the guiding groove of the fresh soil extract sample, so as to realize the quantitative feeding of the fresh soil extract sample. The detection reagent feeding hole 1023 is communicated with the reagent storage groove 20 on the base plate 101 in one-to-one correspondence. After the cover layer 102 of the microfluidic chip is sealed and bonded with the base plate 101, a detection reagent containing a nano-probe material is pre-placed in the reagent storage groove 20 of the base plate 101 through the detection reagent feeding hole 1023. The first air hole 1024 is communicated with the second air hole 17 on the base plate 101, and is configured for balancing the air pressure in each of the first, second, third and fourth flow channel regions of the microfluidic chip. The window 1025 is in one-to-one correspondence with the detection-zone groove 11 on the base plate 101, and is configured as a detection channel of the fluorescence exciter 7 and the receiver 6. By attaching an optically-transparent film, the detection zone is sealed, and the fluorescence detection is improved.

The serpentine groove 14 has a spiral shape, or is formed by a plurality of continuous zigzags.

A middle of a back of the main body of the base plate 101 is provided with a chip fixing hole.

The chip fixing hole is located at the back side of the main body of the base plate 101. The back side of the main body is a side opposite to the surface of the main body on which the soil extract feeding groove 12 is formed. Referring to FIGS. 5 and 9, the side where the chip fixing hole is located is the back side of the main body of the base plate 101. Referring to FIG. 7, the side where the chip fixing hole is located is a lower surface of the main body.

One side of the detection-zone groove 11 is provided with a second air hole 17 communicated with the detection-zone groove 11. The second air hole 17 is arranged in one-to-one correspondence with the first air hole 1024. The second air hole 17 is communicated with the corresponding first air hole 1024.

The window 1025 includes a through hole and an optically-transparent film. The through hole is provided on the main body of the cover plate 102. The optically-transparent film is installed in the through hole.

This application also provides a method for detecting soil nutrients on site by using the microfluidic chip mentioned above, which is performed as follows.

### (Step 1) Installation of the Microfluidic Chip

A microfluidic chip is installed on a centrifugal detector.

### (Step 2) Injection and Flowing of the Fresh Soil Extract Sample

The fresh soil extract sample is injected into the quantitative feeding hole 1021. The centrifugal detector is started to drive the microfluidic chip to rotate at a first rotation speed for T1 seconds. During rotation of the microfluidic chip, the fresh soil extract sample flows from the quantitative feeding hole 1021 sequentially to the soil extract feeding groove 12 and the quantitative feeding groove 18. The excess fresh soil extract sample in the quantitative feeding hole 1021 flows along the guiding groove 1022 to a storage zone 1026 at an end of the guiding groove 1022. In an embodiment, A1 is 200 r/min, and T1 is 30 s.

### (Step 3) Primary Mixing and Reaction

A rotating speed of the centrifugal detector is increased. The centrifugal detector drives the microfluidic chip to rotate at a second rotating speed for T2 seconds. During rotation of the microfluidic chip, the fresh soil extract sample flows from the quantitative feeding groove 18 to the mixing-zone groove 15 through the T-shaped mixing groove 16. A detection reagent flows from the reagent storage groove 20 to the mixing-zone groove 15 through the T-shaped mixing groove 16. The fresh soil extract sample is mixed and reacted with the detection reagent in the mixing-zone groove 15 to obtain a reaction mixture (to-be-detected ions in the fresh soil extract sample is reacted with detection reagent). In an embodiment, A2 is 800 r/min, and T2 is 10 s.

### (Step 4) Secondary Mixing and Reaction

The centrifugal detector stops to allow the reaction mixture to flow from the mixing-zone groove 15 to the serpentine groove 14 to allow further mixing and reaction of the fresh soil extract sample and the detection reagent in the serpentine groove 14, and flow to the capillary micro-valve II 29 between the serpentine groove 14 and the detection-zone groove 11.

### (Step 5) Completion of Mixing and Reaction

The centrifugal detector is restarted to drive the microfluidic chip to rotate at a third rotation speed for T3 seconds to allow the reaction mixture between the serpentine groove 14 and the detection-zone groove 11 to flow into the detection-zone groove 11 through the capillary micro-valve II 29. In an embodiment, A3 is 1500 r/min, and T3 is 20 s.

The number of the reagent storage groove 20 is two or more; and detection reagents for detecting different ions are respectively pre-stored in two or more reagent storage grooves.

Each of the channel branches is provided with a reagent storage groove 20. The detection reagents for detecting different ions are placed in different reagent storage grooves 20 for detecting different ions. The detection reagent includes a probe made of the fluorescent nano material with specific identification capability and a solvent. The probe made of the fluorescent nano material is uniformly dispersed in the solvent, and has specific fluorescence excitation and emission wavelengths. After the detection reagent is mixed with the fresh soil extract sample, the probe made of the nano material in the detection reagent are capable of identifying the to-be-detected ions, leading to rapid and obvious change on fluorescence wavelength. The probe made of the nano material is combined with the detection components to realize in-situ quantitative detection. The probe made of the nano material is prepared through a chemical organic synthesis method. Probes prepared from different ions, having different structures, specific identification capability have high selectivity and capability for visual quantitative detection. The channel branch is selected according to corresponding ion. In this case, multiple ions can be simultaneously detected, improving the detection efficiency.

According to the microfluidic chip and the detection method thereof, the flow control of the fresh soil extract sample and the detection reagent is improved by using a multi-stage capillary microvalve. The reaction and detection are accurately controllable under centrifugal force, and the automatic, simple, accurate and rapid in-situ detection is realized. Consequently, the technical effect brought by rapid and detection on site for acquiring the detection data immediately after injecting a soil sample into a system can be achieved.

In this application, the probe is made of a nano material. The probe is employed in combination with a microfluidic technology to realize synchronously detection of multiple ions in soil. Based on the specific reaction performance of the nano material to the ions and fluorescence changes before and after the reaction, the types and concentrations of multiple ions are obtained. This technology achieves ion specificity identification, which effectively improves synchronous detection of multiple ions in soil. Moreover, the fluorescence detection method can eliminate the interference of visible light band and improve the sensitivity and reliability of ion detection. According to the centrifugal microfluidic chip provided herein, the synchronous detection of various ions in the soil is realized, the types and concentrations of ions in the soil can be efficiently, simply, accurately and quickly obtained, enriching and developing an automatic acquisition and sensing technology of a modern agricultural sensor.

Described above are the basic principles, main features and advantages of this application. It should be understood by those skilled in the art that the above embodiments are only intended to describe principles of this application, and not to limit this application. It should be understood that various changes and improvements of this application made by those skilled in the art based on the content disclosed herein without paying creative effort should be fall within the scope of this application defined by the appended claims.

What is claimed is:

1. A device for detecting soil nutrients on site, comprising:
an extracting grid;
an on-site real-time detection assembly; and
a transfer assembly;
wherein the transfer assembly is configured to transfer a fresh soil extract sample from the extracting grid to the on-site real-time detection assembly;
the on-site real-time detection assembly comprises a driving motor assembly and a detection-analysis assembly, wherein an output shaft of the driving motor assembly is provided with a microfluidic soil chip;
the microfluidic soil chip comprises a base plate; a bottom of the base plate is provided with an aligning slot; the microfluidic soil chip is mounted on the output shaft of the driving motor assembly through the aligning slot; an upper surface of the base plate is located in a housing of the microfluidic soil chip; a center of the upper surface of the base plate is etched with a soil extract feeding groove; a first flow channel region, a second flow channel region, a third flow channel region and a fourth flow channel region are sequentially provided on the base plate from the soil extract feeding groove to outside; and the first flow channel region, the second flow channel region, the third flow channel region and the fourth flow channel region are the same in structure.

2. The device of claim 1, wherein the first flow channel region comprises a quantitative feeding groove for quantitative feeding of a soil extract and a reagent storage groove; the quantitative feeding groove is connected to the soil extract feeding groove through a first microchannel; an outlet of the reagent storage groove is connected to a T-shaped mixing groove through a second microchannel; an outlet of the quantitative feeding groove is connected to the T-shaped mixing groove through a third microchannel; the T-shaped mixing groove is connected to a mixing-zone groove; an outlet of the mixing-zone groove is connected to a detection-zone groove through a serpentine groove; a fluorescence exciter and a receiver are located on a rotation trajectory of the detection-zone groove; and
a width of an end of the second microchannel connected with the T-shaped mixing groove is smaller than a width of an end of the second microchannel connected with the outlet of the reagent storage groove; a width of an end of the third microchannel connected with the T-shaped mixing groove is smaller than a width of an end of the third microchannel connected with the outlet of the quantitative feeding groove;
the serpentine groove is connected to the detection-zone groove through a fourth microchannel, and a width of the fourth microchannel is smaller than the width of the end of the third microchannel connected with the outlet of the quantitative feeding groove.

3. The device of claim 1, wherein the detection-analysis assembly comprises a control processor, an acquisition card, a fluorescence exciter, a receiver and a soil moisture-temperature-electric conductivity sensor; the fluorescence exciter is connected to a first control signal output end of the control processor; the receiver is connected to a first data input end of the control processor through the acquisition card; a data output end of the soil moisture-temperature-electric conductivity sensor is connected to a second data input end of the control processor; and the driving motor assembly is connected to a second control signal output end of the control processor.

4. The device of claim 1, wherein a sealing film is attached to an upper surface of the extracting grid; an extracting reagent is contained in the extracting grid; the number of the extracting grid is n; and any two adjacent extracting grids are connected with each other through a mortise-tenon structure.

5. The device of claim 1, wherein the transfer assembly comprises a negative-pressure suction bag; a rear end of the negative-pressure suction bag is provided with a quick-detachable connector; a front end of the negative-pressure suction bag is provided with a quantitation loop; a front end of the quantitation loop is provided with a suction head; and a filter block is provided in the suction head.

6. The device of claim 2, wherein a reagent storage groove of the first flow channel region is configured to store a specific potassium detection reagent; a reagent storage groove of the second flow channel region is configured to store a specific ammonia-nitrogen detection reagent; a reagent storage groove of the third flow channel region is configured to store a specific nitrate (NO₃⁻) detection reagent; and a reagent storage groove of the fourth flow channel region is configured to store a specific phosphorus detection reagent.

7. The device of claim 2, wherein the base plate is circular; the soil extract feeding groove, the quantitative feeding groove, the reagent storage groove and the mixing-zone groove are circular; and the first flow channel region and the third flow channel region are located on a horizontal axis of the base plate, and the second flow channel region and the fourth flow channel region are located on a vertical axis of the base plate.

8. The device of claim 5, wherein the negative-pressure suction bag and the quick-detachable connector are made of a flexible plastic material; the quantitation loop and the suction head are made of a rigid plastic material; and the filter block is made of filter cotton or quartz sand.

9. The device of claim 1, wherein the microfluidic soil chip further comprises a cover plate located on the base plate.

10. The device of claim 9, wherein the cover plate comprises a main body and a quantitative feeding hole of the fresh soil extract sample; the quantitative feeding hole is provided on a middle of the main body; and the quantitative feeding hole is communicated with the soil extract feeding groove;
the main body is provided with a guiding groove of the fresh soil extract sample, a detection reagent feeding hole and a window; the number of the guiding groove, the number of the detection reagent feeding hole, the number of the window and the number of the first flow channel region, the second flow channel region, the third flow channel region and the fourth flow channel region are the same; the detection reagent feeding hole is communicated with mixing-zone grooves of the first flow channel region, the second flow channel region, the third flow channel region and the fourth flow channel region in one-to-one correspondence; the window is arranged above detection-zone grooves of the first flow channel region, the second flow channel region, the third flow channel region and the fourth flow channel region in one-to-one correspondence; one end of the guiding groove is communicated with the quantitative feeding hole, and the other end of the guiding groove is provided with a storage zone; and a side of the storage zone is provided with an air hole communicated with the storage zone.

11. A method for detecting soil nutrients on site by using the device of claim 1, comprising:
(S91) obtaining a linear relationship curve between soil concentration and fluorescence intensity;
(S92) inserting a soil moisture-temperature-electric conductivity sensor into a field to be analyzed, and acquiring, by a control processor, moisture-temperature-electric conductivity information; wherein the moisture-temperature-electric conductivity information comprises a moisture content t with a unit of %, temperature T with a unit of ° C., and electric conductivity with a unit of mS/cm;
(S93) collecting a fresh soil sample; transferring the fresh soil sample to the extracting grid followed by extraction with an extracting reagent under shaking for 3-5 min to obtain a soil extract sample; wherein a weight ratio of the fresh soil sample to the extracting reagent is 1:5;
(S94) transferring the soil extract sample from the extracting grid to the soil extract feeding groove of the microfluidic soil chip by using the transfer assembly;
(S95) controlling, by the control processor, the driving motor assembly to work to drive the microfluidic soil chip to perform rotating centrifugal motion, so as to allow centrifugal decomposition of the soil extract sample in the microfluidic soil chip;
(S96) controlling, by the control processor, a fluorescence exciter and a receiver to work to acquire fluorescence data of the soil extract sample in a detection-zone groove on the microfluidic soil chip; finding a soil concentration c corresponding to the fluorescence data according to the linear relationship curve; and
(S97) according to the moisture-temperature-electric conductivity information and the soil concentration c, calculating, by the control processor, soil nutrient content with a unit of mg/kg based on the following equation:
Xi=5*c/(1−t);
wherein Xi indicates nitrogen level, phosphorus level or potassium level; 5 is a coefficient, indicating that a weight ratio of the fresh soil sample to water in the extracting grid is 1:5; c is the soil concentration; and t represents the moisture content.

12. The method of claim 11, wherein in step (S95), the centrifugal decomposition is performed through steps of:
(S101) performing a centrifugation at a first rotating speed to allow the soil extract sample to uniformly flow into the first flow channel region, the second flow channel region, the third flow channel region and the fourth flow channel region from the soil extract feeding groove;
(S102) performing a centrifugation at a second rotating speed to allow the soil extract sample and a detection reagent to pass through a T-shaped mixing groove to enter a mixing-zone groove for uniform mixing and reaction to obtain a reaction mixture; wherein the second rotating speed is higher than the first rotating speed;
(S103) subjecting the reaction mixture to standing to allow complete reaction, and allowing the reaction mixture to enter a serpentine groove for further mixing and reaction; and
(S104) performing a centrifugation at a third rotating speed to allow the reaction mixture to pass through the serpentine groove and enter the detection-zone groove; wherein the third rotating speed is higher than the second rotating speed.

13. A microfluidic chip, comprising:
a cover plate and a base plate arranged in sequence;
wherein the base plate comprises a main body, a soil extract feeding groove and a plurality of channel branches; the soil extract feeding groove is provided on a middle portion of a top of the main body of the base plate; the plurality of channel branches are provided on the top of the main body of the base plate, and are uniformly distributed along a periphery of the soil extract feeding groove; the plurality of channel branches comprise a quantitative feeding groove, a reagent storage groove, a mixing-zone groove, a serpentine groove and a detection-zone groove; the quantitative feeding groove is communicated with the soil extract feeding groove; a T-shaped mixing groove is arranged between the quantitative feeding groove and the reagent storage groove; the quantitative feeding groove and the reagent storage groove are connected through the T-shaped mixing groove, and then connected to one end of the mixing-zone groove; the other end of the mixing-zone groove is communicated with one end of the serpentine groove, and the other end of the serpentine mixing-zone groove is connected to the detection-zone groove through a capillary micro-valve.

14. The microfluidic chip of claim 13, wherein the cover plate comprises a main body and a quantitative feeding hole of the fresh soil extract sample; wherein the quantitative feeding hole is arranged on a middle portion of the main body of the cover plate, and is communicated with the soil extract feeding groove; and
the main body of the cover plate is also provided with a guiding groove of the fresh soil extract sample, a detection reagent feeding hole and a window, wherein the number of the guiding groove, the number of the detection reagent feeding hole, the number of the window and the number of the plurality of channel branches are the same; the detection reagent feeding hole is communicated with the mixing-zone groove in one-to-one correspondence the window is arranged above the detection-zone groove in one-to-one correspondence; one end of the guiding groove is communicated with the quantitative feeding hole, and the other end of the guiding groove is provided with a storage zone; and a side of the storage zone is provided with a first air hole communicated with the storage zone.

15. The microfluidic chip of claim 13, wherein the serpentine groove has a spiral shape, or is formed by a plurality of continuous zigzags.

16. The microfluidic chip of claim 13, wherein a middle of a back of the main body of the base plate is provided with a chip fixing hole.

17. The microfluidic chip of claim 14, wherein a side of the detection-zone groove is provided with a second air hole communicated with the detection-zone groove; and the second air hole is communicated with the first air hole in one-to-one correspondence.

18. The microfluidic chip of claim 14, wherein the window comprises a through hole and an optically-transparent film; the through hole is provided on the main body of the cover plate; and the optically-transparent film is provided in the through hole.

19. A method for detecting soil nutrients on site by using the microfluidic chip of claim 14, comprising:
(S1) installing the microfluidic chip on a centrifugal detector;
(S2) injecting the fresh soil extract sample into the quantitative feeding hole; starting the centrifugal detector to drive the microfluidic chip to rotate at a first rotation speed for T1 seconds, wherein during rotation of the microfluidic chip, the fresh soil extract sample flows from the quantitative feeding hole sequentially to the soil extract feeding groove and the quantitative feeding groove and excess fresh soil extract sample in the quantitative feeding hole flows along the guiding groove to the storage zone;
(S3) driving, by the centrifugal detector, the microfluidic chip to rotate at a second rotating speed for T2 seconds, wherein the second rotating speed is larger than the first rotating speed; during rotation of the microfluidic chip, the fresh soil extract sample flows from the quantitative feeding groove to the mixing-zone groove through the T-shaped mixing groove, and a detection reagent flows from the reagent storage groove to the mixing-zone groove through the T-shaped mixing groove; and the fresh soil extract sample is mixed and reacted with the detection reagent in the mixing-zone groove to obtain a reaction mixture;
(S4) stopping the centrifugal detector to allow the reaction mixture to flow from the mixing-zone groove to the serpentine groove to allow further mixing and reaction of the fresh soil extract sample and the detection reagent in the reaction mixture in the serpentine groove, and flow to the capillary micro-valve; and
(S5) restarting the centrifugal detector to drive the microfluidic chip to rotate at a third rotation speed for T3 seconds to allow the reaction mixture to pass through the capillary micro-valve to flow into the detection-zone groove.

20. The method of claim 19, wherein the number of the reagent storage groove is two or more; and detection reagents for detecting different ions are respectively pre-stored in two or more reagent storage grooves.
